(19) United States
(12) Patent Application Publication
(10) Pub. No.: US 2009/0093045 A1
(43) Pub. Date: Apr. 9, 2009

(54) ORGANISM TESTING APPARATUS

(75) Inventors: Kei TAKENAKA, Kashiwa (JP); Yasuhiko SASAKI, Tsuchiura (JP); Ryo MIYAKE, Higashihiroshima (JP); Shigenori TOGASHI, Abiko (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(73) Assignee: Hitachi, Ltd

(21) Appl. No.: 12/244,790

(22) Filed: Oct. 3, 2008

(30) Foreign Application Priority Data
Oct. 3, 2007 (JP) 2007-259330

(51) Int. Cl. C12M 1/34

(52) U.S. Cl. 435/287.1

(57) ABSTRACT

An organism testing apparatus adapted to measure the organisms in a specimen in stable fashion is disclosed in which the organisms are stained, the specimen is concentrated and the information on the organisms contained in the specimen are acquired through a simple process. The apparatus includes a staining unit for staining the organisms having live cells existing in a flowing liquid specimen, a concentration unit for concentrating the organisms in a flowing stained specimen, an individual measuring unit for acquiring the image information on the individuals containing the organisms in the concentrated specimen, and a control unit for measuring the organisms based on the image information on the individuals output from the individual measuring unit.

### BACKGROUND OF THE INVENTION

This invention relates to an organism testing apparatus for measuring the organisms contained in the ballast water, etc.

The ballast water is the sea water loaded as a weight to secure safety when the ship sails without load, and discharged with the arrival at a port.

In recent years, the international movement of the ballast water in the ships operating on an international line has posed the environmental problem that the movement of foreign organisms contained in the ballast water destroys the existing biological system or pollutes the ocean. In Australia, for example, the toxication of cultured shellfishes by toxic plankton is reported.

To cope with this problem, IMO (International Maritime Organization) adopted in February 2004 “International Convention for the Control and Management of Ship's Ballast Water and Sediments” (Ballast Water Management Convention). Under this convention, all the ships using the ballast water are obligated to meet the ballast water discharge standard of the convention in steps.

The ballast water discharge standard provides that the ballast water of one ton is required to contain not more than ten organisms with live cells having the minimum dimension of not less than 50 μm.

The minimum dimension (minimum diameter) of the plankton P1 shown in FIG. 8A, for example, designates the minimum diametric length s1 of the trunk, while the minimum dimension (minimum diameter) of the plankton P2 having legs shown in FIG. 8B similarly designates the minimum diametric length s2 of the trunk. Incidentally, FIGS. 8A, 8B are diagrams showing the dimensions of the plankton.

In a general method currently employed to confirm that the ballast water discharged meets the standard, the ballast water is concentrated to an appropriate concentration and the shape and the life or death of the plankton are judged visually under microscope. This method, which requires both a high inspection cost and a long inspection time, poses the additional problem that the reliability of the test result is dependent on the skill of the inspector. Further, this method is unrealistic in view of the fact that the ballast water of at least 1 m³ is required for the test of the plankton having live cells not less than 50 μm in minimum diameter.

Various measuring instruments aimed at a higher speed and simplification of the measurement of microorganisms in a liquid have been developed in the past. Among them, the imaging flow cytometry for acquiring the shape information of particles such as the cells contained in the urea or blood to be tested has been closely watched as a method capable of directly and quickly measuring the microorganisms in the liquid.

The imaging flow cytometry is a particle measuring method in which the flow diameter of the specimen liquid containing the particles to be tested is reduced to supply the particles one by one for image measurement. The microorganism measuring instrument using this method can acquire the information on the type, shape and size of each microorganism contained in the specimen liquid flowing at the rate of several milliliters per minute.

The method described above is accessible on the internet at Scientific Device Dept., Amco Co., Ltd., “FlowCAM Imaging Flow Cytometer”, as of Jun. 20, 2007.

The imaging flow cytometry is one of the particle measuring methods involving the greatest amount of the specimen liquid tested per unit time. In the case where this method is used for testing the organisms contained in the great amount of the liquid such as the testing of microorganisms in the ballast water requiring at least 1 m³ of the specimen liquid, it is apparent that the concentration before the testing is essential to shorten the processing time. In the concentration process, the organisms in the specimen liquid are highly liable to be physically damaged.

In the testing process using the image measurement, the presence or absence of a body part is one of the criteria for judging the life or death of an organism. The fact that the organism is physically damaged in the concentration process, therefore, deteriorates the reliability of the information on the life or death of the organism. An effective solution to this problem is to employ a concentration method not damaging the organism or to employ a method in which the loss of a body part due to the physical damage in the concentration process has no effect on the life-or-death judgment.

One method conceived to obviate this inconvenience is to stain only the live cells of the organism with a stain before the concentration process, and the presence or absence of the stain may be used as a criterion for life or death of the organism in the test. According to this method, in the case where the loss of a part of the body of the organism is caused in the concentration step, the life or death of the organism can be judged by the presence or absence of the stain.

The staining process executed before concentration is a common practice in testing an organism. The application of this method to the ballast water testing apparatus, however, is accompanied by special problems of the concentration of a great amount of the liquid and a long testing time due to the requirement of the specimen liquid amount of not less than 1 m³.

Even in the case where the processed ballast water of 1 m³ is concentrated at the concentration rate of 1000 and reduced to one liter, several hours are required before completing the test in the following testing step in view of the fact that the amount of the liquid processed by the imaging flow cytometry is several milliliters per min. After concentration of a part of the ballast water, therefore, the standby time of several hours is required before the testing step.

In the organism that has been damaged and killed in the concentration step after being stained, live cells turn dead cells, and the pigment selectively retrieved into the cells of the organism is dispersed out of the cells from micropores of the cell membrane during the testing time. The diameter of the molecules of the pigment used for staining the organism is about a fraction of nm to several nm, and the mean square displacement of the pigment is several tens of μm/sec. Depending on the size and number of the micropores of the cells, the pigment is liable to be dispersed out of the cells and deteriorate the staining state during the standby time before proceeding to the next step. It is thus therefore difficult to judge the information on life or death from the presence or absence of the stain.

### SUMMARY OF THE INVENTION

In view of the present situation described above, the object of this invention is to provide an organism testing apparatus in which the organisms contained in the specimen such as the ballast water can be measured in stable fashion by acquiring the information on the staining of the organisms, the concentration of the specimen and the types of the contained organisms through a simple process.

In order to achieve the object described above, according to this invention, there is provided an organism testing apparatus comprising a staining unit for staining the organisms having live cells existing in a flowing liquid specimen, a concentration unit for concentrating the flowing stained specimen to increase the concentration of the organisms, an individual measuring unit for acquiring the image information on the individuals including the organisms in the concentrated specimen, and a control unit for measuring the organisms based on the image information of the individuals output from the individual measuring unit.

According to this invention, the step of staining the organisms in the liquid specimen the step of concentrating the organisms in the liquid and the step of acquiring the information on the organisms in the liquid can be executed in an integrated flow system. As compared with the method in which each step is executed in a batch, therefore, the standby time before a part of the specimen that has finished a given step proceeds to the next step can be remarkably shortened or reduced to zero. Thus, the deterioration of the staining state which otherwise might occur during the standby time can be prevented, and the stable information on the life or death of the organisms can be acquired.

Other objects, features and advantages of the invention will become apparent from the following description of the embodiments of the invention taken in conjunction with the accompanying drawings.

### BRIEF DESCRIPTION OF THE DRAWINGS

FIG. 1 is a diagram showing a basic system of the organism testing apparatus according to an embodiment of the invention.

FIG. 2A is a plan view schematically showing the internal structure of the flow-type staining unit of the organism testing apparatus, and FIG. 2B a sectional view of the flow-type staining unit as taken in line A-A in FIG. 2A.

FIG. 3 is a sectional view schematically showing the internal structure of the flow-type concentration unit of the organism testing apparatus.

FIG. 4 is a sectional view schematically showing the internal structure of the flow-type particle measuring unit of the organism testing apparatus.

FIGS. 5A, 5B and 5C are diagrams showing the judgment data for judging the life or death of a plankton constituting one of the particles.

FIG. 6 is a flowchart showing the control method of the control unit executed upon completion of measurement by the organism testing apparatus.

FIG. 7 is a flowchart showing the concentration rate control method for the organism testing apparatus.

FIGS. 8A and 8B are diagrams showing the shape and size of the plankton.

### DETAILED DESCRIPTION OF THE INVENTION

An embodiment of the invention is explained below with reference to the accompanying drawings.

The organism testing apparatus 1 according to an embodiment of the invention is a device for counting the number of organisms such as plankton having live cells and not less than 50 μm in minimum diameter in a liquid specimen of not less than 1 m³. Incidentally, the minimum diameter of an organism having live cells is defined as the diameter of the part shortest in size of the trunk of the particular organism.

The measurement by counting the organisms having live cells in the organism testing apparatus 1 according to this embodiment is explained in detail.

### <<Configuration of Organism Testing Apparatus 1>>

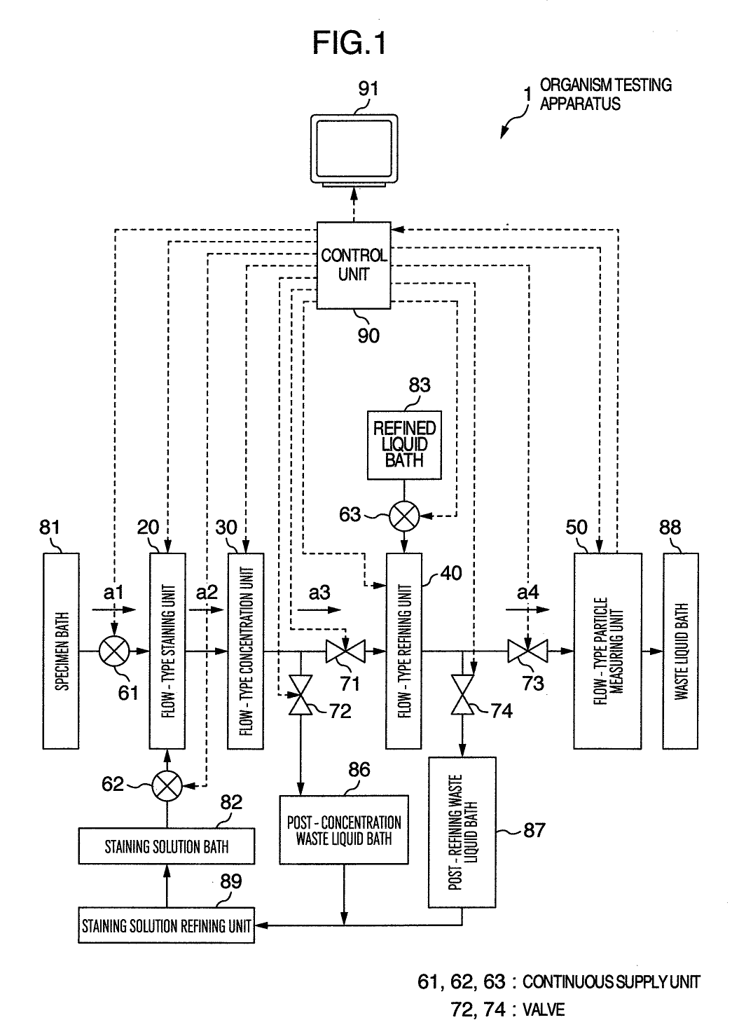

FIG. 1 is a basic system diagram showing the organism testing apparatus 1 according to this invention.

As shown in FIG. 1, the organism testing apparatus 1 includes, as essential parts for executing the process in each step, a flow-type staining unit 20 for mixing, while pouring in a flow, a specimen and a staining solution for staining the organisms having live cells in the specimen, a flow-type concentration unit 30 for concentrating the flowing specimen containing the organisms having a predetermined range of size, a flow-type refining unit 40 for removing the organisms in other than the predetermined range of size from the flowing specimen, and a flow-type particle measuring unit 50 for acquiring the image information on the particles in the flowing specimen.

The process in each step of the organism testing apparatus 1 is executed in an integrated flow process without any waiting time, and the specimen to be measured stored in a specimen liquid bath 81, as indicated by arrows a1, a2, a3, a4, flows from the specimen liquid bath 81 through the flow-type staining unit 20, the flow-type concentration unit 30, the flow-type refining unit 40 and the flow-type particle measuring unit 50 in that order thereby to make a predetermined measurement.

The organism testing apparatus 1 is equipped with liquid baths for storing the liquids such as the staining solution to be used in the aforementioned process, including a specimen liquid bath 81 for storing the specimen before testing, a staining solution bath 82 for storing the staining solution used in the flow-type staining unit 20, and a refining liquid bath 83 for storing the refining liquid used for refining in the flow-type refining unit 40.

The organism testing apparatus 1 is also equipped with waste liquid baths for storing the liquids to be disposed of after the process, including a post-concentration waste liquid bath 86 for storing the liquid to be discharged from the flow-type concentration unit 30, a post-refining waste liquid bath 87 for storing the liquid containing the particles containing the organisms removed by the flow-type refining unit 40 and a post-measurement waste liquid bath 88 for storing the waste liquid of the specimen of which the particle measurement is finished by the flow-type particle measuring unit 50.

The organism testing apparatus 1 is further equipped with a continuous supply unit for continuously feeding the specimen, the staining solution and the refining liquid, including a specimen continuous supply unit 61 for supplying the specimen continuously to the flow-type staining unit 20 from the specimen bath 81, a staining solution continuous supply unit 62 for continuously supplying the staining solution to the flow-type staining unit 20 from the staining solution bath 82 and a refining liquid continuous supply unit 63 for continuously supplying the refining liquid to the flow-type refining unit 40 from the refining liquid bath 83. Incidentally, instead of the pumps used as the continuous supply unit 61, 62, 63 according to this embodiment, any machine other than the pump which can feed the liquids may be used with equal effect.

Also, as valves for controlling the flow rate of the supplied liquids such as the specimen and the staining solution, the organism testing apparatus 1 is equipped with, as shown in FIG. 1, a valve 71 arranged between the concentration unit 30 and the refining unit 40 to control the flow rate of the liquid passing through the tube connecting the flow-type concentration unit 30 and the flow-type refining unit 40, a valve 72 arranged between the concentration unit 30 and the post-concentration waste liquid bath 86 to control the amount of the liquid passing through the tube connecting the flow-type concentration unit 30 and the post-concentration waste liquid bath 86, a valve 73 arranged between the refining unit 40 and a particle measuring unit 50 to control the amount of the liquid passing through the tube connecting the flow-type refining unit 40 and the flow-type particle measuring unit 50, and a valve 74 arranged between the refining unit 40 and the post-refining waste liquid bath 87 for controlling the liquid passing through the tube connecting the flow-type refining unit 40 and the post-refining waste liquid bath 87.

The organism testing apparatus 1 described above has a control unit 90 as a control unit for controlling, by outputting a control signal, etc. to, the units 20, 30, the units 40, 50, the continuous supply units 61, 62, 63 and the valves 71, 72, 73, 74 while at the same time analyzing the particle information obtained by the flow-type particle measuring unit 50. Also, the analysis result of the control unit 90 is output by an output unit 91 such as a liquid crystal display or a printer.

Incidentally, the organism testing apparatus 1 has a staining solution refining unit 89 for refining and reusing the used staining solution contained in the waste liquid discharged by the post-concentration waste liquid bath 86 and the post-refining waste liquid bath 87. The staining solution dissolved in the waste liquid, after being refined by a reverse osmosis (RO) membrane, is returned to the staining solution bath 82 and reused.

Incidentally, the staining solution refining unit 89 may be separated into two systems of a first staining solution refining unit for refining and reusing the used staining solution from the waste liquid discharged by the post-concentration waste liquid bath 86 and a second staining solution refining unit for refining and reusing the used staining solution contained in the waste liquid discharged from the post-refining waste liquid bath 87.

### <<Process in Each Step of Organism Testing Apparatus 1>>

Next, the process executed in each step of the organism testing apparatus 1 is explained.

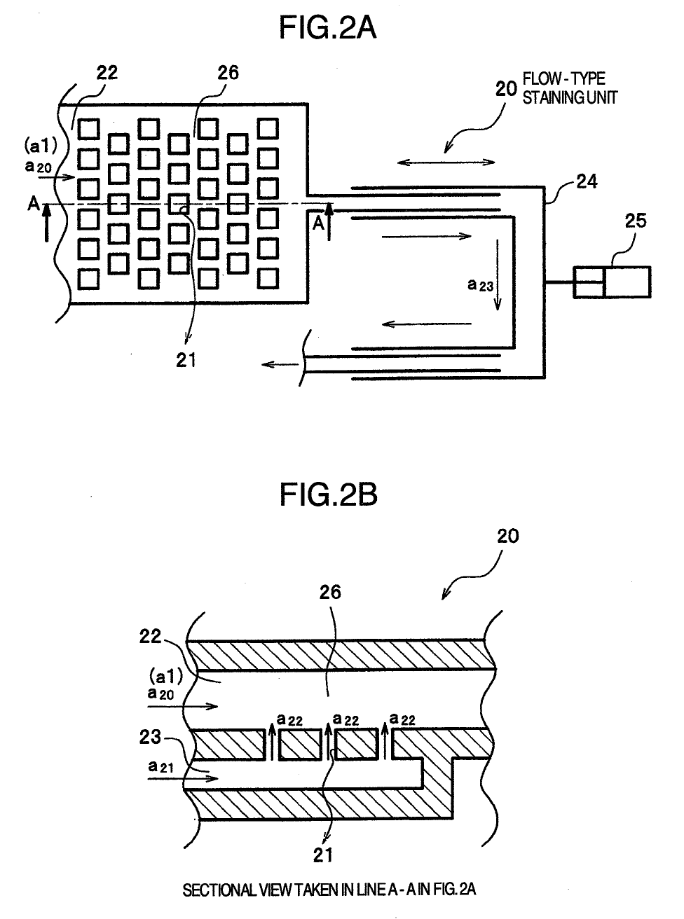

FIG. 2A is a plan view schematically showing the internal structure of the flow-type staining unit 20, and FIG. 2B a sectional view taken in line A-A in the flow-type staining unit 20 shown in FIG. 2A.

As shown in FIG. 1, the specimen stored in the specimen bath 81 containing the specimen to be measured is supplied to the flow-type staining unit 20 from the specimen bath 81 as indicated by arrow a1 using the specimen continuous supply unit 61, and as shown in FIGS. 2A, 2B, flows into the staining bath 26 as indicated by arrow a20 from the specimen inlet 22.

The staining solution for staining the organisms having live cells, on the other hand, is supplied to the flow-type staining unit 20 by the staining solution continuous supply unit 62 from the staining solution bath 82 shown in FIG. 1, and from the staining solution inlet 23 shown in FIG. 2B, flows into the flow-type staining unit 20 as indicated by arrow a21.

As shown in FIG. 2B, the staining solution that has flowed into the flow-type staining unit 20 from the staining solution inlet 23, as indicated by arrow a21, flows into the staining bath 26 along the direction of arrow a22 through plural staining solution ejection ports 21, 21, . . . , and comes to be mixed with the specimen in the staining bath 26. In this way, the specimen and the staining solution, after being mixed in the staining bath 26, flows in the direction along arrow a23 through a mixed flow tube 24 as shown in FIG. 2A, while the organisms having live cells in the specimen are stained by the staining solution.

The staining time of the organisms having live cells in the specimen can be appropriately adjusted by moving a part of the mixed flow tube 24 and thus changing the total length of the mixed flow tube 24 by a mixed flow tube drive unit 25 such as a hydraulic cylinder thereby to change the flowing time before reaching the adjacent flow-type concentration unit 30 (FIG. 1) on downstream side.

The mixed flow tube drive unit 25, in place of the hydraulic cylinder shown, may of course be a mechanism for moving the mixed flow tube 24 using the reduction mechanism by means of the drive force of a motor or any of various mechanisms that can change the total length of the mixed flow tube 24 by moving a part thereof.

According to this embodiment, the mixing ratio between the specimen and the staining solution is set to the staining solution of 5 g versus the specimen of 1000 liters, or in other words, the staining solution of 1 g is mixed with the specimen of 200,000 milliliters for five minutes. This mixing time is automatically determined by the control unit 90 in accordance with the state of the specimen. For example, a specimen containing a well-known organism having live cells is supplied, and the staining degree thereof is obtained from the image information of the flow-type particle measuring unit 50, so that the mixing time may be determined automatically by the control unit 90.

As the staining solution, a pigment such as neutral red or methyl blue that is selectively retrieved into the live cells, i.e. the pigment that stains only the live cells is used.

As described above, a two-liquid mixture containing the specimen stained in the flow-type staining unit 20 through the mixing flow tube 24 as indicated by arrow a23 in FIG. 2A, flows downstream into the flow-type concentration unit 30 as indicated by arrow a2 in FIG. 1.

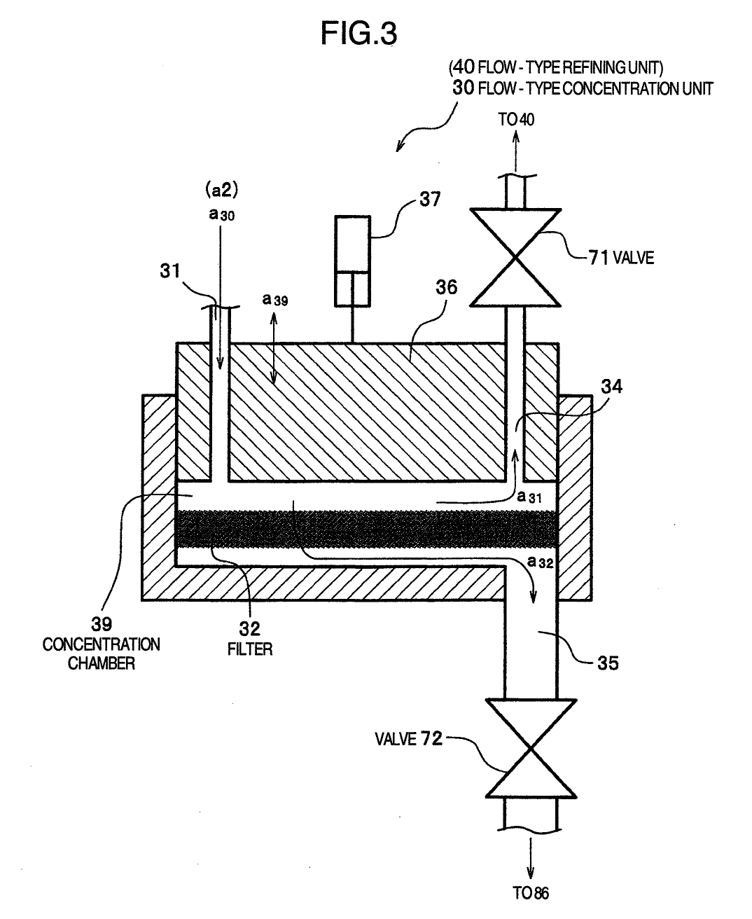

FIG. 3 is a sectional view schematically showing the internal structure of the flow-type concentration unit 30.

As shown in FIG. 3, the specimen stained by the flow-type staining unit 20 flows into the flow-type concentration unit 30 along the direction of arrow a30 from the flow-type concentration unit inlet 31, and is separated into two streams in the terms of the ratio of the liquid amount passing through the valve 71 between the flow-type concentration unit 30 and the flow-type refining unit 40 (FIG. 1) and the liquid amount passing through the valve 72 between the flow-type concentration unit 30 and the post-concentration waste liquid bath 86 (FIG. 1). Then, the specimen flows out separately from the concentrated liquid outlet 34 and the filtered liquid outlet 35.

Between the flow-type concentration unit inlet 31 and the filter liquid outlet 35, a filter 32 having the bore of 50 μm is arranged, so that the particles larger than 50 μm that have flowed into the flow-type concentration unit 30 are blocked by the filter 32 and flow to the concentrated liquid outlet 34 along the direction of arrow a31, while the particles smaller in size than 50 μm that have flowed into the flow-type concentration unit 30, on the other hand, flows to the filtered liquid outlet 35 through the filter 32 as indicated by arrow a32.

The concentration rate of the specimen in the flow-type concentration unit 30 is determined by the ratio between the amount of the liquid passing through the valve 71 arranged between the flow-type concentration unit 30 and the flow-type refining unit 40 and the amount of the liquid passing through the valve 72 arranged between the flow-type concentration unit 30 and the post-concentration waste liquid bath 86.

Incidentally, according to this embodiment, the concentration rate is set to 1000 times richer. Nevertheless, the concentration rate is automatically set to an optimum value by the feedback operation of the control unit 90 in accordance with the prevailing state of the specimen. This feedback control operation is carried out by reason of the fact that the staining degree is changed with the state of the ballast water constituting the specimen, and therefore, if the staining degree is excessive, even the dead organisms would be stained. Incidentally, the feedback operation can be conducted by the flow-type particle measuring unit 50 acquiring the image information and the control unit 90 judging the staining degree based on the image information.

The concentration rate of 1000 times richer can be realized, for example, by forming ten stages of the flow-type concentration unit 30 of FIG. 3 having the passing liquid amount ratio of 1:10 between the valve 71 arranged between the flow-type concentration unit 30 and the flow-type refining unit 40 (FIG. 1) and the valve 72 arranged between the flow-type concentration unit 30 and the post-concentration waste liquid bath 86 (FIG. 1).

Also, according to this embodiment, the flow-type concentration unit 30 having the filter 32 of the bore 50 μm is employed. As an alternative, plural flow-type concentration units having different bores may be arranged in parallel in the organism testing apparatus 1 and switched in accordance with the state of the specimen.

As shown in FIG. 3, a part of the particles flowing in the flow-type concentration unit 30 is adsorbed on the filter 32. Since the flow indicated by arrow a31 in the direction of the filtering surface of the filter 32, i.e. the flow in the direction along the filtering surface of the filter 32 has the effect of removing the particles adsorbed on the filter 32, however, the reduction in the number of particles due to adsorption to the filter 32 can be suppressed.

In the case where the amount of the specimen flowing into the flow-type concentration unit 30 is reduced, the flow velocity along the filtering surface (direction along arrow a31) of the filter 32 is reduced, thereby reducing the force to remove the particles adsorbed on the filter 32.

Nevertheless, the flow velocity of the specimen can be maintained at a constant value by moving the capacity variable wall 36 as indicated by arrow a39 using the variable wall drive unit 37 such as the hydraulic cylinder and thus reducing the capacity of the flow-type concentration unit 30.

Incidentally, the variable wall drive unit 37 may be replaced with a mechanism for moving the capacity variable wall 36 using the reduction mechanism with the motor drive force without using the hydraulic cylinder shown. In this way, any of the various mechanisms which can change the capacity of the flow-type concentration unit 30 by moving the capacity variable wall 36 can of course employed.

The filtered liquid flowing in the direction shown by arrow a32 in FIG. 3 after the filter 32 of the flow-type concentration unit 30, on the other hand, is disposed into the post-concentration waste liquid bath 86 (FIG. 1) through the valve 72 arranged between the flow-type concentration unit 30 and the post-concentration waste liquid bath 86.

Then, as shown in FIG. 1, the specimen containing the particles larger than 50 μm that have passed through the flow-type concentration unit 30, as indicated by arrow a3, flows into the flow-type refining unit 40 through the valve 71 arranged between the flow-type concentration unit 30 and the flow-type refining unit 40. This flow-type refining unit 40 has a similar structure to the flow-type concentration unit 30 (FIG. 3).

In the flow-type refining unit 40, the specimen containing the particles larger than 50 μm that have passed through the flow-type concentration unit 30 is supplied with the refined liquid not containing the particles from the refined liquid bath 83, so that the concentration of all the particles is once reduced, and after removing extraneous dust and particles through the filter, the specimen is concentrated again. Thus, the ratio of the particles larger than the bore of the filter 32 is relatively increased. In other words, the specimen is refined by increasing the purity of the particles larger than 50 μm in the specimen.

The liquid filtered through the filter in the flow-type refining unit 40, on the other hand, as shown in FIG. 1, is disposed into the post-refining waste liquid bath 87 through the valve 74 arranged between the flow-type refining unit 40 and the post-refining waste liquid bath 87.

In this way, the specimen refined in the flow-type refining unit 40, as indicated by arrow a4 in FIG. 1, flows out of the flow-type refining unit 40 and flows into the flow-type particle measuring unit 50 through the valve 73 arranged between the flow-type refining unit 40 and the flow-type particle measuring unit 50.

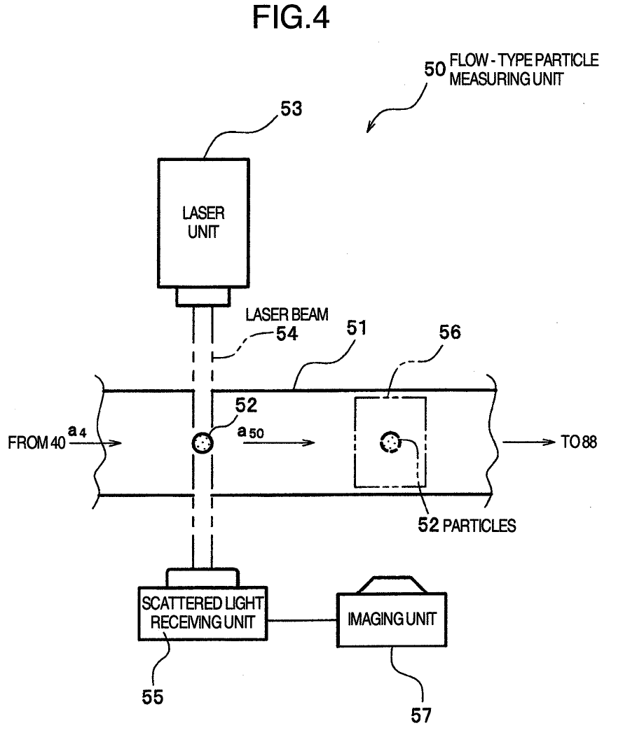

FIG. 4 is a sectional view schematically showing the configuration of the flow-type particle measuring unit 50.

As shown in FIG. 4, the flow-type particle measuring unit 50 includes a laser unit 53 for outputting a laser beam 54 to be applied to the particles in the specimen, a scattered light receiving unit 55 for receiving the laser beam 54 applied to the particles in the specimen and an imaging unit 57 for imaging the particles that have flow in the imaging range 56.

The specimen that has passed through the flow-type refining unit 40 shown in FIG. 1, as indicated by arrow a4 in FIG. 4, flows into the flow-type particle measuring unit 50, and flows as indicated by arrow a50 through the measuring path 51. While the specimen flows in this way, the measurement is conducted on the specimen as described below.

First, the particles 52 flowing into the imaging range 56 are detected using the laser unit 53 and the scattered light receiving unit 55. Specifically, the scattered light generated from the particles 52 when passing across the laser beam 54 output from the laser unit 53 is detected by the scattered light receiving unit 55 thereby to detect the passage of the particles 52.

The time t required for the specimen to flow from the position of the laser beam 54 to the imaging range 56 (indicated by dashed line in FIG. 4) is determined by the velocity of the specimen. By acquiring the image of the imaging range 56 after the time t determined by the flow velocity, therefore, the image information of the particles 52 that have entered the imaging range 56 can be picked up and acquired by the imaging unit 57. Incidentally, the passage of the particles 52 may alternatively be detected by an electrical unit such as the coal-tar counter.

The time when the particles 52 pass through the laser beam 54 is proportional to the size of the particles 52 since all the particles 52 pass through the laser beam 54. Therefore, an autozoom mechanism for adjusting the size of the imaging range in accordance with the passage time is included in the imaging unit 57.

Incidentally, the particles 52, if elongate in shape, receive the force of the specimen flow and therefore flow along the specimen flow. In other words, the specimen flows in the same direction as the longitudinal direction of the particles 52.

As a result, the signal containing the information on each particle 52 in the specimen passing through the laser beam 54 is input from the scattered light receiving unit 55 by the control unit 90 (FIG. 1). Thus, the control unit 90 can analyze the maximum length of the particle 52 from the information on the time when the particle 52 passes through the laser beam 54.

Then, the control unit 90 controls the operation of the autozoom mechanism for automatically adjusting the size of the imaging range 56, i.e. the size of the visual field of the imaging unit 57 in accordance with the time when the particle 52 passes, i.e. the maximum length of the particle 52. Thus, the imaging operation corresponding to the object to be measured becomes possible.

In this way, after the time t calculated from the time point of detection of passage of the particle 52 to the time point when the particle 52 flows into the imaging range 56, the imaging unit 57 operates and acquires the image information of the particle 52 in the specimen that has flowed into the imaging range 56 through the measurement flow path 51.

Then, the acquired image information is output to the control unit 90 as an image information signal from the imaging unit 57. The life or death of the organism, i.e. the particle 52 is judged in accordance with the presence or absence of the stain and the size of the particle 52 is measured from the image information acquired by the control unit 90.

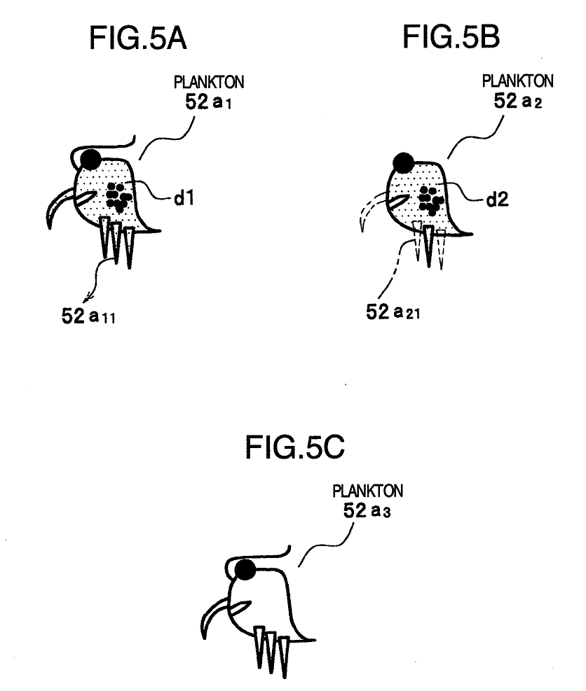

FIGS. 5A, 5B and 5C are diagrams showing the judgment data for judging the life or death of the plankton 52a1, 52a2, 52a3 constituting the organisms as the particles 52.

Assume that the plankton constituting one of the particles in the image information is the plankton 52a1 stained (indicated by dots in the trunk) as shown in FIG. 5A or the plankton 52a2 stained (indicated by dots in the trunk) as shown in FIG. 5B. In these cases, the plankton is judged as live. In the case of the plankton 52a3 not stained (no dots in the trunk) as shown in FIG. 5C, on the other hand, the plankton is judged as dead.

Incidentally, as compared with 52a11 shown in FIG. 5A, the loss of the legs 52a21 indicated by two-dot chains of the plankton 52a2 shown in FIG. 5B is caused at the time of concentration in the flow-type concentration unit 30 or the flow-type refining unit 40.

### <<Control Method at the End of Measurement>>

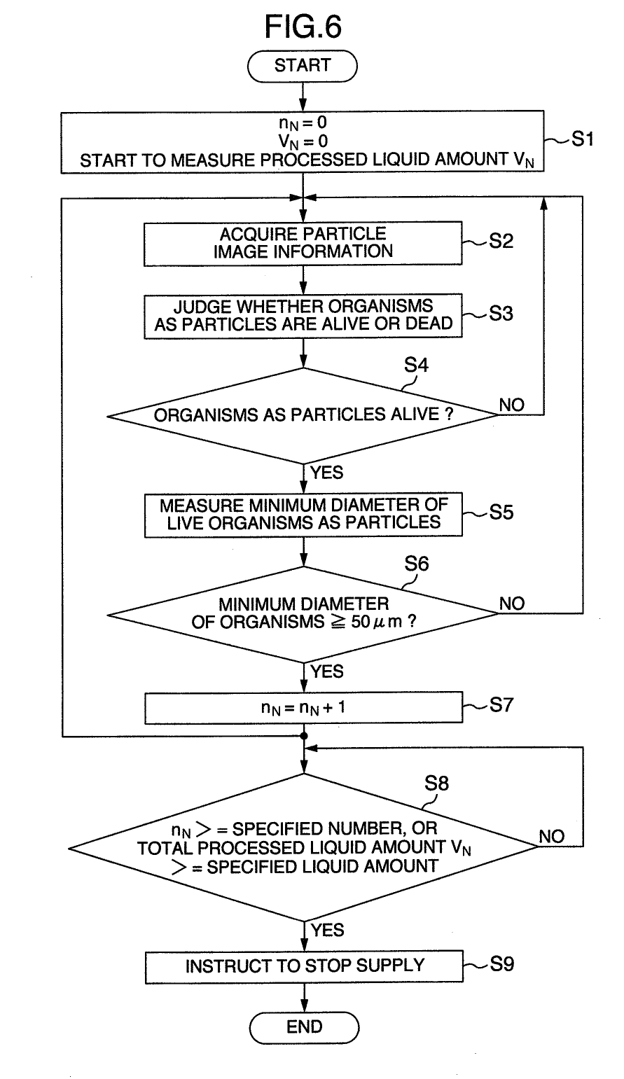

Next, the control method by the control unit 90 (FIG. 1) at the end of measurement by the organism testing apparatus 1 is explained with reference to FIG. 6. Incidentally, FIG. 6 is a flowchart showing the control method of the control unit 90 at the end of measurement.

First, in step S1 of FIG. 6, the total processed liquid amount V_{N}, or for example, the variable V_{N} indicating the total processed amount of the ballast water is set to zero. Also, the variable n_{N} indicating the number of the particles 52 having live cells and having the specified range of size is set to 0. Incidentally, the specified range, though defined as the size of organisms having the minimum diameter of not less than 50 μm, can of course be set arbitrarily.

At the same time, the control unit 90 starts to measure the total processed liquid amount V_{N} for calculation thereof based on the liquid amount supplied by the continuous supply unit 61 per unit time.

Incidentally, the total processed liquid amount V_{N} or, for example, the total processed amount of the ballast water may be measured by a flow rate sensor (not shown) arranged in the pipe between the specimen bath 81 and the continuous supply unit 61.

Then, in step S2 of FIG. 6, the image information of a particle 52 in the specimen is acquired using the flow-type particle measuring unit 50 as shown in FIG. 4.

After that, in step S3 of FIG. 6, based on the image information of the particle 52 obtained from the flow-type particle measuring unit 50, the control unit 90 judges whether the organism constituting the particle 52 is alive or dead. The life or death of the organism constituting the particle 52 is judged by, for example, the method shown in FIGS. 5A, 5B and 5C.

Then, step S4 of FIG. 6 judges whether the organism constituting the particle 52 in the image information acquired is alive or dead, i.e. whether the organism has live cells or not.

In the case where the organism constituting the particle 52 in the acquired image information is not alive, i.e. the organism has no live cells (NO in step S4 of FIG. 6), on the other hand, the measurement of the organisms is stopped and the control proceeds to step S2 in FIG. 6.

In the case where the organism constituting the particle 52 in the acquired image information is alive, i.e. the organism has live cells (YES in step S4 of FIG. 6), the control unit 90 measures the minimum diameter of the organism constituting the particle 52 based on the image information of the particle 52 obtained from the flow-type particle measuring unit 50 in step S5 of FIG. 6.

Incidentally, the minimum diameter of the organism constituting the particle 52 is defined as the length of the shortest diameter of the trunk in the contour of the organism.

The minimum diameter of the plankton 52a1 shown in FIG. 5A, for example, is defined as the length of the shortest diameter in the contour of the trunk d1 of the plankton 52a1, while the minimum diameter of the plankton 52a2 shown in FIG. 5B, on the other hand, is defined as the length of the shortest diameter of the trunk d2 of the plankton 52a2.

The length of the shortest diameter of the trunk d1 shown in FIG. 5A may be the size of the trunk d1 in the direction perpendicular to the page of FIG. 5A. Similarly, the length of the shortest diameter of the trunk d2 shown in FIG. 5B may be the size of the trunk d2 in the direction perpendicular to the page of FIG. 5B.

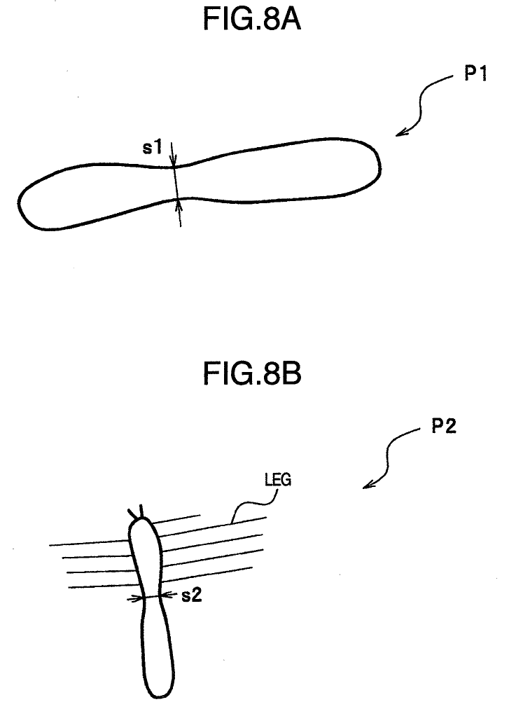

The minimum diameter of the plankton P1 shown in FIG. 8A indicates the length s1 of the shortest diameter of the trunk, while the minimum diameter of the plankton P2 having legs shown in FIG. 8B indicates the length s2 of the shortest diameter of the trunk.

Then, step S6 of FIG. 6 judges whether the minimum diameter of the live organism as the particle 52 is not less than 50 μm or not.

In the case where the minimum diameter of the live organism as the particle 52 is less than 50 μm (NO in step S6 of FIG. 6), the measurement of the organism is stopped and the control proceeds to step S2 in FIG. 6.

In the case where the minimum diameter of the live organism as the particle 52 is not less than 50 μm (YES in step S6 of FIG. 6), on the other hand, step S7 of FIG. 6 calculates the number n_{N}=n_{N}+1 of the particles having live cells and having the specified range of size, and thus counts the number of particles having live cells and having the specified range of size.

Then, the control proceeds to step S2 in FIG. 6, where the process of steps S2 to S7 is executed again.

At the same time, step S8 of FIG. 6 judges whether n_{N} is not less than a specified number or the total processed liquid amount V_{N} is not less than a specified liquid amount. Incidentally, the specified number is, for example, 10, and the total processed liquid amount V_{N} is, for example, one ton of ballast water.

In the case where n_{N} is less than the specified number and the past total processed liquid amount V_{N} is less than the specified liquid amount (NO in step S8 of FIG. 6), the control proceeds to step S2 in FIG. 6 to continue the measurement, and through steps S2 to S7, the judgment of S8 in FIG. 6 is repeated.

In the case where n_{N} is not less than the specified number or the past total processed liquid amount V_{N} is not less than the specified liquid amount (YES in step S8 of FIG. 6), on the other hand, the control unit 90 judges that the test is finished in step S9 of FIG. 6, and sends a stop command to the specimen continuous supply unit 61 and the staining solution continuous supply unit 62 and so on thereby to end the measurement.

Also in the case where the measurement is continued by proceeding to step S2 in FIG. 6 and the judgment of step S8 in FIG. 6 is repeated, if judged YES in step S8 of FIG. 6, the control proceeds to step S9 in FIG. 6. Thus, the control unit 90 judges that the test is finished, and sends a stop command to the specimen continuous supply unit 61 and the staining solution continuous supply unit 62 and so on thereby to end the measurement.

### <<Concentration Rate Control Method>>

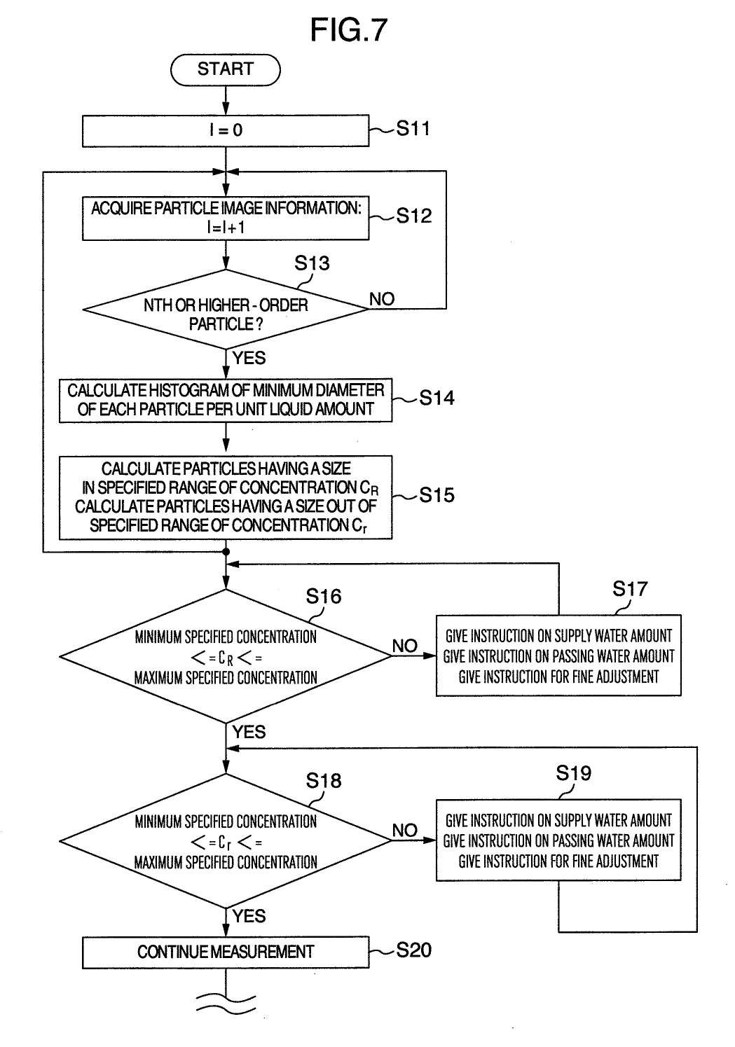

Next, the concentration rate control method using the control unit 90 in the organism testing apparatus 1 is explained with reference to FIG. 7. FIG. 7 is a flowchart of the concentration rate control method.

First, in step S11 of FIG. 7, the variable for counting the Nth particle 52, i.e. the variable I indicating the number of times the image information is acquired is set to zero. Incidentally, N is an arbitrary positive integer for preparing a histogram (frequency distribution chart) and can be set arbitrarily.

Then, in step S12 of FIG. 7, the flow-type particle measuring unit 50 acquires the image information of the particles 52, and performs, each time the image information is acquired, the arithmetic operation I=I+1 for counting the number I of times the image information is acquired. Incidentally, the arithmetic operation described above may be performed by sorting the acquired image information on the particles 52 according to the size, etc. of the particles 52.

Then, step S13 in FIG. 7 judges whether the flow-type particle measuring unit 50 has counted the N or more particles 52.

In the case where the particles 52 counted by the flow-type particle measuring unit 50 is less than N (NO in step S13 of FIG. 7), the control proceeds to step S12 of FIG. 7.

In the case where the particles 52 counted by the flow-type particle measuring unit 50 reaches N (YES in step S13 of FIG. 7), on the other hand, the histogram (frequency distribution chart) of the minimum diameter of the particles 52 per unit liquid amount is determined from the information on the minimum diameter of each particle 52 and the passed liquid amount v before counting the Nth particle 52 from the (N−a)th particle 52 in step S14 of FIG. 7. Incidentally, the character a is an arbitrary positive integer smaller than N and adapted to prepare a histogram with the effective frequency indicated by (N−a). Also, the minimum diameter of the particle 52, as described above, is the length of the shortest diameter of the trunk in the contour of the particle 52.

Now, the passed liquid amount v may be measured by a flow rate sensor (not shown) arranged immediately before the flow-type particle measuring unit 50 or, in the control unit 90, may be calculated using the liquid amount supplied by the continuous supply units 61, 62, 63 per unit time and the liquid amount passed through the valves 72, 73, 74 per unit time.

Then, in step S15 of FIG. 7, the concentration C_{R} of the particles 52 having a size in the specified range of the minimum diameter and the concentration Cᵣ of the particles 52 out of the specified range of the minimum diameter are calculated from the histogram thus obtained. Incidentally, the specified range, though defined as the minimum diameter of not less than 50 μm according to this embodiment, may of course be determined arbitrarily.

Then, the control proceeds to step S12 in FIG. 7, where the process of steps S12 to S15 is repeatedly executed.

At the same time, step S16 of FIG. 7 judges whether the concentration C_{R} of the particles having a size in the specified range of the minimum diameter is not lower than the minimum specified concentration but not higher than the maximum specified concentration or not.

In the case where the concentration C_{R} of the particles having a size in the specified range of the minimum diameter is lower than the minimum specified concentration or higher than the maximum specified concentration, i.e. in the case where the concentration C_{R} of the particles having a size in the specified range of the minimum diameter is out of a predetermined range of the measurement conditions (NO in step S16 of FIG. 7), then, in order to set C_{R} within a predetermined range, the control unit 90 (FIG. 1) gives an instruction to the continuous supply units 61, 62, 63 (FIG. 1) on the amount of the liquid to be supplied and an instruction to the valves 71, 72, 73, 74 (FIG. 1) on the amount of the liquid to be passed in step S17 of FIG. 7. Also, the control unit 90 instructs the flow-type concentration unit 30 (FIGS. 1, 3) to change the concentration rate and the flow-type refining unit 40 to change the refining rate.

In the case where the particle concentration C_{R} is lower than the minimum specified concentration, for example, the continuous supply units 61, 62, 63 are instructed to supply more liquid, and the valves 71, 72, 73, 74 to pass more liquid. Also, the capacity of the concentration chamber 39 is reduced by the variable wall drive unit 37 in the flow-type concentration unit 30 shown in FIG. 3. Further, the concentration rate is adjusted upward, for example, by increasing the refining rate of the flow-type refining unit 40.

In the case where the concentration C_{R} of the particles having a size in the specified range of the minimum diameter exceeds the maximum specified concentration, on the contrary, the continuous supply units 61, 62, 63 are instructed to supply less liquid, and the valves 71, 72, 73, 74 to pass less water. Also, the capacity of the concentration chamber 39 is increased by the variable wall drive unit 37 in the flow-type concentration unit 30 shown in FIG. 3. Further, the concentration rate is adjusted downward, for example, by decreasing the refining rate of the flow-type refining unit 40.

After executing the process of step S17 shown in FIG. 7, the control is passed to step S16 in FIG. 7.

In the case where the concentration C_{R} of the particles having a size in the specified range of the minimum diameter is not lower than the minimum specified concentration but not higher than the maximum specified concentration in step S16 of FIG. 7, i.e. in the case where the value C_{R} is within a predetermined range of the measurement conditions (YES in step S16 of FIG. 7), on the other hand, the control proceeds to step S18 in FIG. 7 to judge whether the concentration Cᵣ of the particles out of the specified range of the minimum diameter is not lower than the minimum specified concentration but not higher than the maximum specified concentration or not.

Incidentally, according to this embodiment, the specified range is defined as not less than the minimum diameter of 50 μm as described above.

In the case where the concentration Cᵣ of the particles out of the specified range of the minimum diameter is lower than the minimum specified concentration or higher than the maximum specified concentration, i.e. in the case where the value Cᵣ is out of a predetermined range of the measurement conditions (NO in step S8 of FIG. 7), the control proceeds to step S19 in FIG. 7. Then, in order to set the value Cᵣ within the predetermined range, the control unit 90 (FIG. 1) gives an instruction to the continuous supply units 61, 62, 63 on the amount of the liquid to be supplied, to the valves 71, 72, 73, 74 (FIG. 1) on the amount of the liquid to be passed and to the flow-type refining unit 40 (FIGS. 1, 3) to change the refining rate. Then, the control proceeds to step S18 in FIG. 7 to repeat the judgment of step S18.

In the case where the concentration Cᵣ of the particles out of the specified range of the minimum diameter is not lower than the minimum specified concentration but not higher than the maximum specified concentration in step S18 of FIG. 7, i.e. in the case where the value Cᵣ is within the predetermined range of the measurement conditions (YES in step S18 of FIG. 7), on the other hand, the control proceeds to step S20 in FIG. 7 to continue the measurement.

The method of controlling the concentration rate in the organism testing apparatus 1 is described above.

With this configuration, the staining step, the concentration step and the step of acquiring the information on the organisms including those in the liquid specimen are automated, and therefore, both the job load on the inspection worker and the effect of the skill of the worker on the measurement result can be reduced, while at the same time making it possible to obtain a stable measurement result.

Further, the respective steps are integrated as a flow system, and therefore, as compared with the conventional method in which each step is executed in a batch, the standby time before part of ballast water that has finished one step proceeds to the next step can be reduced to zero, i.e. eliminated. As a result, the staining state of the stained organism is prevented from being deteriorated during the standby time, and stable information on life or death can be acquired.

Incidentally, this embodiment is explained taking organisms such as plankton or objects other than organisms as an example of the particles contained in the specimen. The individuals described in the appended claims, however, include a wide variety of organisms and objects other than organisms larger than the particles contained in the specimen. In addition to the plankton described above, the larva of the crab, the fish such as baby sardines and other larger organisms including aquatic mollusks such as octopus are some examples.

Incidentally, this embodiment refers to a case including the flow-type refining unit 40. The flow-type refining unit 40, which is only for increasing the concentration of the organisms to be measured, may be done without.

Also, in the case where a larger object such as the crab larva is to be measured, the meshes of the filter 32 of the flow-type concentration unit 30 and the filter of the flow-type refining unit 40 can be enlarged, and as described above, the imaging operation with a larger visual field of the lens can be performed using the autozoom mechanism of the imaging unit 57.

Incidentally, in place of the autozoom mechanism of the imaging unit 57, plural lenses having different visual field ranges may be switched appropriately in accordance with the size of the object to be measured. Also, the imaging unit 57 may be configured of plural lenses of different visual fields and the autozoom mechanism combined to increase the flexibility as an imaging unit.

Also, according to this embodiment, the organisms are measured by measuring the minimum diameter thereof and judging the life or death of the organisms. As an alternative, other sizes than the minimum diameter of the organisms or other measurement than the judgment of life or death of the organisms may be used with equal effect.

### Various Embodiments

The configurations described below can be implemented.

A first organism testing apparatus includes at least a staining unit for staining the organisms having live cells existing in a flowing liquid specimen, a concentration unit for increasing the concentration of the organisms in the flow of a stained specimen, an individual measurement unit for acquiring the image information on the individuals containing the organisms in the concentrated specimen, and a control unit for measuring the organisms based on the image information on the individuals output from the individual measurement unit.

With the configuration of the first organism testing apparatus, the organisms can be measured in a flow system without killing the organisms having live cells, and the deterioration of the staining state which otherwise might occur during the standby time is prevented, thereby making possible a variety of stable measurement of organisms.

In a second organism testing apparatus, based on the first organism testing apparatus, the organism measurement can include both the organism shape measurement and the judgment of life or death of the organisms from the staining state.

With the configuration of the second organism testing apparatus, the measurement is possible to judge whether the international convention on the ballast water is complied with or not.

A third organism testing apparatus, based on the first organism testing apparatus, includes a refining unit arranged between the concentration unit and the individual measurement unit to further increase the concentration of organisms after supplying the refined liquid containing no organisms in a flowing specimen sent from the concentration unit.

In the configuration of the third organism testing apparatus, the provision of the refining unit can increase the concentration of the organisms to be measured.

A fourth organism testing apparatus, based on the first organism testing apparatus, includes a specimen transport unit for conveying the specimen through the staining unit, the concentration unit and the individual measurement unit in that order and a transport control unit for controlling the conveyance and stop of the specimen on the specimen transport unit.

With the configuration of the fourth organism testing apparatus, the specimen can be conveyed or stopped as required.

A fifth organism testing apparatus, based on the third organism testing apparatus, includes a flow rate control unit arranged between the concentration unit and the refining unit to control the flow rate of the specimen from the concentration unit to the refining unit and a first flow rate control unit for controlling the flow rate control unit.

With the configuration of the fifth organism testing apparatus, the flow rate from the concentration unit to the refining unit can be controlled.

A sixth organism testing apparatus, based on the third organism testing apparatus, includes a flow rate control unit arranged between the refining unit and the individual measuring unit to control the flow rate from the refining unit to the individual measuring unit and a second flow rate control unit for controlling the flow rate control unit.

With the configuration of the fifth organism testing apparatus, the flow rate from the refining unit to the individual measuring unit can be controlled.

In a seventh organism testing apparatus, based on the first organism testing apparatus, the concentration unit concentrates the organisms not less than 50 μm in diameter.

With the configuration of the seventh organism testing apparatus, the organisms having the minimum diameter of not less than 50 μm can be concentrated and measured.

With an eighth organism testing apparatus, based on the third organism testing apparatus, the refining unit removes the organisms having the maximum diameter of less than 50 μm.

With the configuration of the eighth organism testing apparatus, the refining unit can remove the organisms having the maximum diameter of less than 50 μm and thus increase the concentration of the organisms having the maximum diameter of not less than 50 μm.

In a ninth organism testing apparatus, based on the first organism testing apparatus, the amount of the specimen is set to not less than 1 m³.

The configuration of the ninth organism testing apparatus makes it possible to concentrate the ballast water of not less than 1 ton, for example, to 1000 times richer and measure the organisms with the specimen amount of not less than 1 m³.

A tenth organism testing apparatus, based on the first organism testing apparatus, includes a flowing amount acquisition unit for determining the flowing amount of the specimen, wherein the control unit counts the number of organisms having the minimum diameter of not less than 50 μm with live cells and the measurement is ended in the case where ten organisms are counted before the flowing amount of the specimen acquired by the flowing amount acquisition unit reaches 1 m³.

With the configuration of the tenth organism testing apparatus, the specimen such as the ballast water of 1 ton is concentrated to 1000 times richer, for example, and the measurement can be made to judge whether at least 10 organisms having the minimum diameter of 50 μm with live cells exist in the specimen of one ton.

An 11th organism testing apparatus, based on the first organism testing apparatus, includes a flowing amount acquisition unit for determining the flowing amount of the specimen, wherein the control unit measures the frequency of the minimum diameter of the individuals per unit liquid amount acquired by the flowing amount acquisition unit thereby to determine the concentration rate of the concentration unit from the particular frequency.

In the configuration of the 11th organism testing apparatus, the frequency of the minimum diameter of the individuals per unit liquid amount of the specimen is measured and the concentration rate of the concentration unit is determined from the particular frequency. Thus, the concentration rate can be determined in accordance with the frequency of the minimum diameter of the individuals per unit specimen liquid amount thereby to measure the organisms.

A 12th organism testing apparatus, based on the third organism testing apparatus, includes a flowing amount acquisition unit for determining the flowing amount of the specimen, wherein the control unit measures the frequency of the minimum diameter of individuals per unit liquid amount acquired by the flowing amount acquisition unit thereby to determine the refining rate of the refining unit from the particular frequency.

In the configuration of the 12th organism testing apparatus, the frequency of the minimum diameter of the individuals per unit liquid amount acquired by the flowing amount acquisition unit is measured and the refining rate of the refining unit is determined from the particular frequency. Therefore, the concentration of the specimen can be increased for measurement in accordance with the frequency of the minimum diameter of the individuals per unit specimen liquid amount.

A 13th organism testing apparatus, based on the first organism testing apparatus, includes a first staining solution refining unit for recovering the staining solution supplied to the specimen from the staining unit in the liquid discharged as a waste from the concentration unit.

In the configuration of the 13th organism testing apparatus, the staining solution in the liquid discharged as a waste from the concentration unit is recovered by the first staining solution refining unit, and therefore, the staining solution can be effectively used.

A 14th organism testing apparatus, based on the third organism testing apparatus, includes a second staining solution refining unit for recovering, from the liquid discharged as a waste by the refining unit, the staining solution supplied to the specimen from the staining unit.

In the configuration of the 14th organism testing apparatus, the staining solution in the liquid discharged as a waste from the refining unit is recovered by the second staining solution refining unit, and therefore, the staining solution can be effectively used.

It should be further understood by those skilled in the art that although the foregoing description has been made on embodiments of the invention, the invention is not limited thereto and various changes and modifications may be made without departing from the spirit of the invention and the scope of the appended claims.

1. An organism testing apparatus comprising:
a staining unit for staining an organism having a live cell in a flowing liquid specimen and;
a concentration unit for increasing concentration of the organism in the stained flowing specimen;
an individual measuring unit for acquiring image information of an individual containing the organism in the concentrated specimen; and
a control unit for measuring the organism based on the image information on the individual output from the individual measuring unit.

2. The organism testing apparatus according to claim 1,
wherein the control unit measures the organism by judging whether the organism is alive or dead based on measurement of a shape of the organism and presence or absence of a staining state of the organism.

3. The organism testing apparatus according to claim 1, further comprising:
a refining unit arranged between the concentration unit and the individual measurement unit to supply a refined liquid not containing the organism and then concentrate the specimen flowing from the concentration unit thereby to further increase the concentration of the organism.

4. The organism testing apparatus according to claim 1, comprising:
a specimen transport unit for transporting the specimen through the staining unit, the concentration unit and the individual measuring unit in that order; and
a transport control unit for controlling a transport and stop of the specimen by the specimen transport unit.

5. The organism testing apparatus according to claim 3, comprising:
a flow rate control unit arranged between the concentration unit and the refining unit to control the flow rate of the specimen from the concentration unit to the refining unit; and
a first flow rate control unit for controlling the flow rate control unit.

6. The organism testing apparatus according to claim 3, comprising:
a flow rate control unit arranged between the refining unit and the individual measuring unit to control a flow rate from the refining unit to the individual measuring unit; and
a second flow rate control unit for controlling the flow rate control unit.

7. The organism testing apparatus according to claim 1,
wherein the concentration unit concentrates the organism having a size of a minimum diameter of not less than 50 μm.

8. The organism testing apparatus according to claim 3,
wherein the refining unit removes the organism having a size of a maximum diameter of less than 50 μm.

9. The organism testing apparatus according to claim 1,
wherein an amount of the specimen is not less than 1 m³.

10. The organism testing apparatus according to claim 1, comprising:
a flowing amount acquisition unit for determining a flowing amount of the specimen;
wherein the control unit counts a number of the organism having a size of a minimum diameter of not less than 50 μm and having the live cell, and ends a measurement in a case where ten of the organism are counted before the flowing amount of the specimen acquired by the flowing amount acquisition unit reaches 1 m³.

11. The organism testing apparatus according to claim 1, comprising:
a flowing amount acquisition unit for determining a flowing amount of the specimen;
wherein the control unit measures a frequency of a minimum diameter of the individual per unit liquid amount acquired by the flowing amount acquisition unit and determines a concentration rate of the concentration unit based on a particular frequency.

12. The organism testing apparatus according to claim 3, comprising:
a flowing amount acquisition unit for determining a flowing amount of the specimen;
wherein the control unit measures a frequency of a minimum diameter of the individual per unit liquid amount acquired by the flowing amount acquisition unit and determines a refining rate of the refining unit based on a particular frequency.

13. The organism testing apparatus according to claim 1, comprising:
a first staining solution refining unit for recovering, from a liquid discharged as a waste by the concentration unit, a staining solution supplied to the specimen by the staining unit.

14. The organism testing apparatus according to claim 3, comprising:
a second staining solution refining unit for recovering, from a liquid discharged as a waste by the refining unit, a staining solution supplied to the specimen by the staining unit.
